(19) United States
(12) Patent Application Publication
(10) Pub. No.: US 2022/0202940 A1
(43) Pub. Date: Jun. 30, 2022

(54) METHOD OF STABILIZING A DIALYSIS SOLUTION

(71) Applicant: Fresenius Medical Care Deutschland GmbH, Bad Homburg (DE)

(72) Inventors: Andreas LAUBROCK, Wehrheim (DE); Jochen HUPPERT, Saarbrücken (DE); Pascal MATHIS, Saarwellingen (DE); Robert BERLICH, St. Wendel (DE)

(73) Assignee: Fresenius Medical Care Deutschland GmbH, Bad Homburg (DE)

(21) Appl. No.: 17/260,652

(22) PCT Filed: Jul. 16, 2019
(86) PCT No.: PCT/EP2019/069070

§ 371 Date: Jan. 15, 2021

(30) Foreign Application Priority Data
Jul. 17, 2018 (DE) 10 2018 117 264.8

(51) Int. Cl. A61K 47/24; A61K 33/10; A61K 9/08; A61K 9/00

(52) U.S. Cl. CPC A61K 47/24; A61K 9/0026; A61K 9/08; A61K 33/10

(57) ABSTRACT

The present invention relates to a method of stabilizing a dialysis solution that includes calcium ions and bicarbonate ions, wherein a phosphate and/or an organic phosphate ester is added to the dialysis solution at a distance of time from its preparation.

The invention relates to a method for a subsequent stabilization of a dialysis solution with respect to a precipitation of calcium carbonate.

It is known from WO 2016/041634 A1 that small amounts of orthophosphate in the millimolar concentration range can prevent the precipitation of sparingly soluble calcium carbonate from dialysis solutions. It is further known from WO 2016/202462 A1 that organic phosphates such as glycerophosphate can exert a stabilizing effect, as can mixtures of orthophosphates and organic phosphates. The stabilization takes place in this respect in the prior art by an additional addition of a defined phosphate quantity on the preparation of a solution.

A number of dialysis solutions available on the market do not include any phosphates as stabilization agents and are thus prone to precipitation reactions.

It is the object of the invention to provide a method to reduce the susceptibility of such solutions to unwanted precipitation reactions.

Against this background, the invention relates to a method of stabilizing a dialysis solution that includes calcium ions and bicarbonate ions, wherein a phosphate and/or an organic phosphate ester is added to the dialysis solution at a distance of time from its preparation.

The invention therefore has as its subject the time-delayed addition (spiking) of a phosphate or of an organic phosphate ester to stabilize existing dialysis solutions. It has surprisingly been found that a more stabilizing effect against the precipitation of calcium carbonate can be achieved not only by an initial addition, but also by a subsequent addition of a phosphate or of an organic phosphate ester. The subsequent addition of the phosphate thus inhibits further precipitation events despite the already present germs. A dissolution of existing precipitate was not observed.

The term “dialysis solution” is to be given a broad interpretation and covers all the solutions that can be used during a dialysis treatment, that is, in addition to solutions for hemodialysis, for example also substitution solutions or solutions for peritoneal dialysis. The use of the method in accordance with the invention is, however, particularly preferred in solutions for continuous renal replacement therapy (CRRT), in particular substitution solutions. Substitution solutions are infused directly into the blood of the patient and may therefore not include any particles.

The term dialysis solution in accordance with the understanding of the present disclosure also covers those dialysis solutions that are still present in the form of two or more separate individual solutions whose mixing results in the obtaining of a ready-to-use dialysis solution. Such individual solutions can be present, for example, in a dual chamber bag. Provision can thus be made within the framework of the method in accordance with the invention to spike an individual solution with a phosphate or with an organic phosphate ester. An addition to an individual solution can be preferred here that includes bicarbonate ions and/or no calcium ions.

Where phosphates or phosphate esters are spoken of in the present disclosure, this term always covers both the completely protonated acids and the partially protonated acids and the salts. The addition of salts, in particular sodium salts of the phosphates or phosphate esters, is particularly preferred within the framework of the invention.

Provision is made in an embodiment that the phosphate is an orthophosphate and/or that the organic phosphate ester is an organic ester of the orthophosphate.

Provision is made in an embodiment that the organic ester of the orthophosphate is a glycerol orthophosphate. This substance is already established as an active substance, for example, for parenteral nutrition and is also monographed in the European Pharmocopoeia (January 2009:1995). This relatively small molecule can be metabolized quickly while releasing orthophosphate. The glycerol-orthophosphate can be a glycerol-2-orthophosphate, a glycerol-3-orthophosphate or a mixture thereof.

Provision is made in an embodiment that the phosphate is added in an amount of up to 0.4 mmol/l. An addition of up to 0.375 mmol/l, up to 0.25 mmol/l, or up to 0.2 mmol/l can be preferred. Provision is made in an embodiment that the phosphate is added in an amount of at least 0.05 mmol/l. Such phosphate concentrations are below physiological concentration values so that the medical action of the dialysis solution is not influenced.

Provision is made in an embodiment that the organic phosphate ester is added in an amount of up to 1.25 mmol/l. An addition of up to 1.2 mmol/l can be preferred. Provision is made in an embodiment that the organic phosphate ester is added in an amount of at least 0.8 mmol/l. An addition of at least 1.0 mmol/l can be preferred. A phosphate concentration of 0.8 to 1.25 mmol/l, and preferably from 1 to 1.2 mmol/l, corresponds to a concentration which can be used to regulate the phosphate balance of dialysis patients and to prevent hypophosphatemia, for example. A physiologically desirable phosphate concentration with a simultaneous increase in stability with respect to precipitation reactions of calcium carbonates can thus subsequently be set in the case of phosphate-free dialysis solutions by a subsequent addition of organic phosphate esters and in particular of glycerophosphate.

Provision is made in an embodiment that both a phosphate and an organic phosphate ester are added to the solution at a distance of time from its preparation, with provision preferably being made that the concentration ratio between the phosphate and the organic phosphate ester is between 0.3/0.7 and 0.9/0.1. On the addition of both an organic ester of phosphoric acid and of orthophosphate, a synergistic effect can occur which can result in an even better stabilization of the solution with respect to the precipitation of calcium carbonate.

Provision is made in an embodiment that the distance of time from the preparation amounts to more than 30 minutes, more than 60 minutes, more than 90 minutes, or more than 105 minutes. Even aged solutions having an already elevated pH can therefore be subsequently stabilized and thus made safer within the framework of a method in accordance with the invention.

Provision is made in an embodiment that the distance of time from the preparation amounts to more than one week, more than one month, more than six months, or more than one year. Even aged solutions having an already elevated pH can therefore be subsequently stabilized and thus made safer within the framework of a method in accordance with the invention.

Provision is made in an embodiment that the pH of the solution at the time of the subsequent addition is greater than 7.2, greater than 7.4, or greater than 7.6. The pH of freshly prepared dialysis solutions is typically lower and can, for example, be between approximately 7.0 and 7.6. The pH of the solution at the time of the subsequent addition should, however, still be below 8.0 where possible since calcium carbonate has frequently already precipitated to a significant degree at a pH of 8.0 and the addition would thus no longer be effective.

In an embodiment, the dialysis solution also includes further electrolytes in addition to the calcium ions, preferably sodium ions, potassium ions, magnesium ions and/or chloride ions. Magnesium can also precipitate as a sparingly soluble carbonate and this precipitation reaction can also be potentially inhibited by means of the phosphate or the phosphate ester.

In an embodiment, the dialysis solution or the substitution furthermore includes at least one osmotic agent, for example a saccharide such as glucose or a derivate therefrom.

Said solution components in the dialysis solution can, for example, be present independently of one another in the concentrations given in Table 1.

TABLE 1

| | |
|---|---|
| Calcium ions: | 1-2 mmol/l, for example 1.5 mmol/l |
| Magnesium ions: | 0.2-0.8 mmol/l, for example 0.5 or 0.75 mmol/l |
| Potassium ions: | Up to 8, and preferably up to 4 mmol/l |
| Sodium ions: | 120-160 mmol/l, for example 140 mmol/l |
| Bicarbonate ions (incl. | 30-40 mmol/l, for example 35 mmol/l |
| carbonate ions and | |
| dissolved CO₂): | |
| Osmotic agent: | 4-12 mmol/l, for example 5.6 mmol/l |
| Chloride ions: | 100-120 mmol/l, for example 109 mmol/l |

Further details and advantages of the invention result from the experiments and embodiments described in the following with reference to the Figures. There are shown in the Figures:

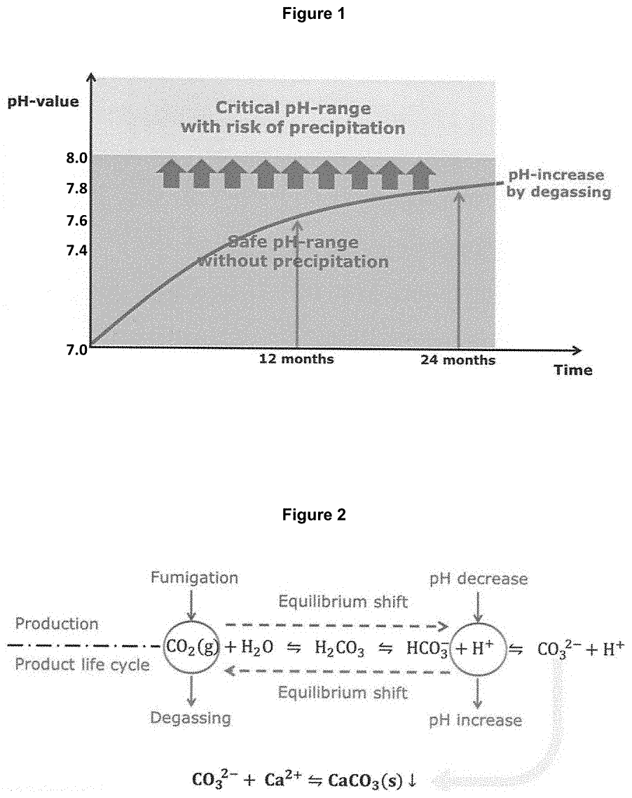

FIG. 1: an exemplary development of the pH of a dialysis solution over its life;

FIG. 2: a schematic representation of chemical processes during the preparation and during the life cycle of a dialysis solution;

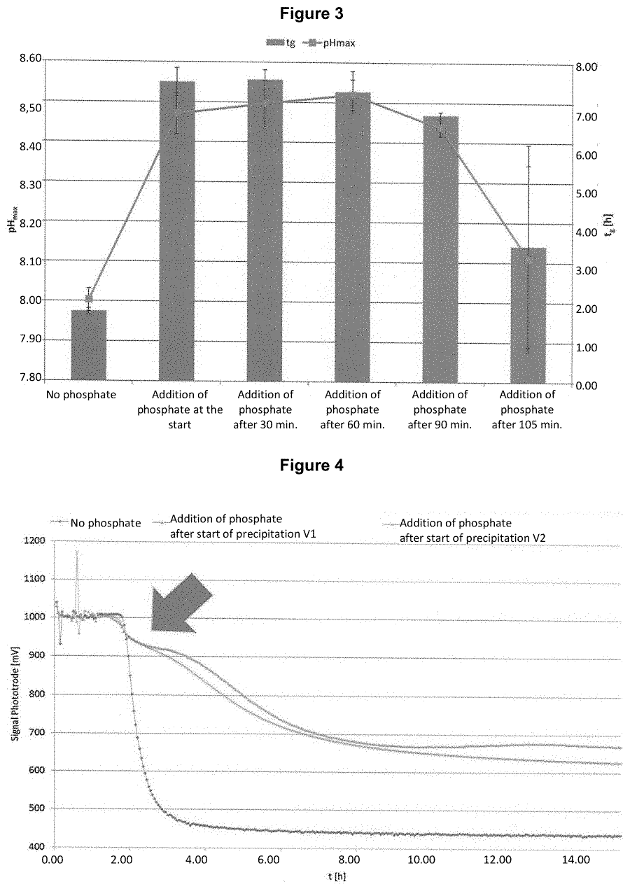

FIG. 3: Values for pHmax and tG in the experiment of Example 1;

FIG. 4: Phototrode signal for selected data points in the experiment of Example 1:

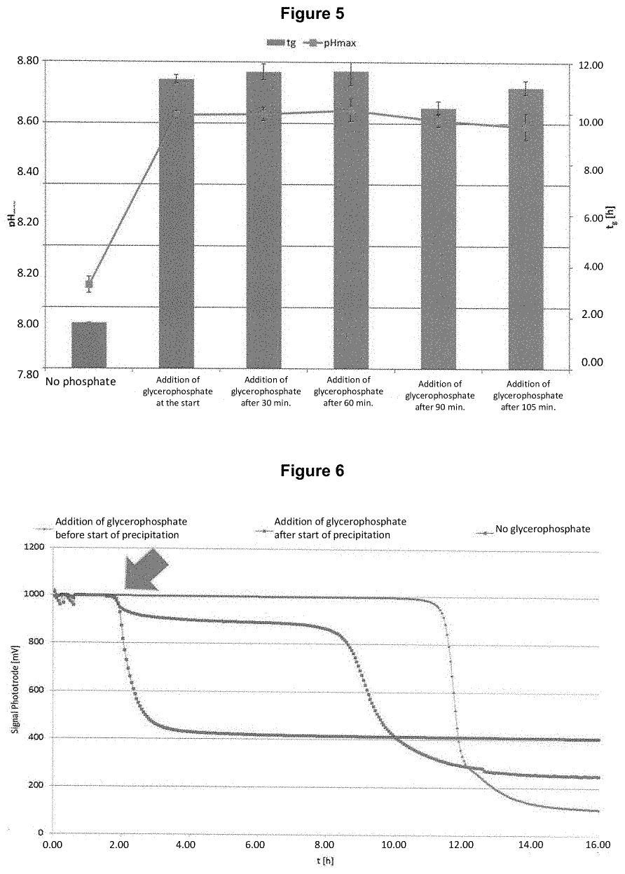

FIG. 5: Values for pHmax and tG in the experiment of Example 2; and

FIG. 6: Phototrode signal for selected data points in the experiment of Example 2.

The pH of the solution is increased by the CO₂ degassing from a bicarbonate-buffered dialysis solution, which promotes the precipitation of calcium carbonate in the case of solutions containing calcium. The pH at which a significant precipitation of calcium carbonate starts is a criterion for the stability of the solution and is subsequently called pHmax. The higher the value for pHmax, the more stable the solution. The time at which the significant precipitation of calcium carbonate starts is subsequently called tG to represent the “time of germination”. An exemplary development of the pH of a dialysis solution over its life is shown in FIG. 1, with pHmax in this illustration being assumed as 8.0 and with tG not yet having been reached in the time period shown.

The stabilizing effect of a subsequent addition of phosphate or of a phosphate ester to existing dialysis solutions was examined using the rapid degassing method in a carousel setup. THz rapid degassing method in this variant allows an accelerated aging of dialysis solutions by degassing the CO₂ from the bicarbonate buffer. The degassing of CO₂ that normally occur slowly during the life of the products can be simulated within a few hours with this experiment setup.

The chemical processes in the preparation and in the degassing of the solution are shown schematically in FIG. 2. CO₂ is thus introduced into the solution in the preparation to reduce the pH in the solution. This process gradually reverses during the life cycle due to degassing processes, whereby, with a resulting higher pH, an unwanted precipitation of calcium carbonate begins from a certain point onward.

### EXAMPLE 1

The dialysis solution DUOSOL 4551 of B. Braun is exposed to a simulated aging at 40° C. in a plurality of experiments with and without the addition of orthophosphate as part of the above-described rapid degassing method. The dialysis solution DUOSOL 4551 containing calcium does not contain any phosphates as stabilization agents and is therefore susceptible to precipitation reactions.

After a specific time period, 250 μl of a parent solution having 100 mmol/l orthophosphate is added to a plurality of flasks containing 250 ml DUOSOL 4551, which results in a concentration of 0.1 mmol/l of orthophosphate in the flask. Three separate experiments are carried out for each data point and the mean value is used. The results with respect to pHmax and tG are shown in FIG. 3. The phototrode signal for selected data points is shown in FIG. 4.

Without any addition of orthophosphate, a precipitation of calcium carbonate begins after approximately two hours, which can be recognized from the decrease of the transmission of FIG. 4. The addition of orthophosphate after the start of the precipitation has an inhibiting effect. A dissolution of this precipitate is not observed. An addition after the start of precipitation therefore slows down the further precipitation process.

It can be seen from FIG. 3 that there is no interaction between the stabilizing effect and the time of the addition of orthophosphate. The large standard deviation that can be recognized in FIG. 3 for the measurement point at 105 minutes results from the fact that a significant precipitation had already started on two of the three repetitions of the experiment.

### Example 2

The experiment of Example 1 is repeated in the same manner with the only difference that glycerophosphate is added instead of orthophosphate this time. That is, after a specific time period, 2.5 ml of a parent solution having 100 mmol/l glycerophosphate is added to the flasks containing 250 ml DUOSOL 4551, which results in a concentration of 1.0 mmol/l of glycerophosphate in the flask. The results with respect to pHmax and tG are shown in FIG. 5. The phototrode signal for selected data points is shown in FIG. 6. Similar conclusions can be drawn from these graphs as were made for Example 1 with reference to the graphs of FIGS. 3 and 4.

In summary, it can be stated that the time-delayed addition of phosphates or of phosphate esters to bicarbonate buffered dialysis solutions containing calcium has the same stabilizing effect with respect to a precipitation of calcium carbonate as an initial addition during the preparation of the solution. Even aged solutions having an elevated pH can be stabilized and thus made safer by this procedure. This principle works before and also during a dialysis treatment.

In the case of organic phosphate esters and in particular of glycerophosphate, the addition of around 1 mmol/l enables the setting of a physiological phosphate concentration with a simultaneous increase in stability with respect to precipitation reactions of calcium carbonates.

1. A method of stabilizing a dialysis solution that includes calcium ions and bicarbonate ions,
characterized in that
a phosphate and/or an organic phosphate ester is added to the dialysis solution at a distance of time from its preparation.

2. A method in accordance with claim 1, characterized in that the phosphate is an orthophosphate; and/or in that the organic phosphate ester is an organic ester of the orthophosphate.

3. A method in accordance with claim 2, characterized in that the organic ester of the orthophosphate is a glycerol orthophosphate.

4. A method in accordance with claim 1, characterized in that the phosphate is added in an amount of up to 0.4 mmol/l.

5. A method in accordance with claim 1, characterized in that the phosphate is added in an amount of at least 0.05 mmol/l.

6. A method in accordance with claim 1, characterized in that the organic phosphate ester is added in an amount of up to 1.25 mmol/l.

7. A method in accordance with claim 1, characterized in that the organic phosphate ester is added in an amount of at least 0.8 mmol/l.

8. A method in accordance with claim 1, characterized in that both a phosphate and an organic phosphate ester are added to the solution at a distance of time from its preparation, with provision preferably being made that the concentration ratio between the phosphate and the organic phosphate ester is between 0.3/0.7 and 0.9/0.1.

9. A method in accordance with claim 1, characterized in that the distance of time from the preparation amounts to more than 30 minutes, more than 60 minutes, more than 90 minutes, or more than 105 minutes.

10. A method in accordance with claim 1, characterized in that the distance of time from the preparation amounts to more than one week, more than one month, more than six months, or more than one year.

11. A method in accordance with claim 1, characterized in that the pH of the solution at the time of the subsequent addition is greater than 7.2, greater than 7.4, or greater than 7.6
